(19) United States
(12) Patent Application Publication
(10) Pub. No.: US 2021/0315800 A1
(43) Pub. Date: Oct. 14, 2021

(54) COSMETIC OR DERMATOLOGICAL COMPOSITIONS

(71) Applicant: BCR, Lege-Cap-Ferret (FR)

(72) Inventors: Francois CANAC, Auteuil le Roi (FR); Philippe REVELEAU, Lege-Cap-Ferret (FR); Jean Julien BARONNET, Studio City, CA (US)

(21) Appl. No.: 17/264,497

(22) PCT Filed: Jul. 30, 2019
(86) PCT No.: PCT/EP2019/070440

§ 371 Date: Jan. 29, 2021

(30) Foreign Application Priority Data
Jul. 30, 2018 (FR) 1857081

(51) Int. Cl. A61K 8/98; A61Q 19/08; A61Q 19/02; A61K 35/618

(52) U.S. Cl. CPC A61K 8/987; A61Q 19/08; A61K 2800/92; A61K 35/618; A61K 2236/333; A61Q 19/02

(57) ABSTRACT

The invention relates to an alkaline hydrolysate of oyster meat, and to the cosmetic and dermatological uses thereof for treating signs of ageing such as wrinkles or blemishes or for improving the firmness of the skin and achieving a skin-firming effect.

The present invention relates to a novel extract of oyster, in particular of oyster meat, and to pharmaceutical and cosmetic compositions containing same, more particularly those intended for topical application, their preparation process and their uses in dermatology or cosmetology, in particular in the treatment of skin disorders such as changes in skin texture, skin color, skin transparency, irritation and the appearance of wrinkles.

Skin aging results from two distinct and independent processes that involve intrinsic or extrinsic factors. Extrinsic aging corresponds to aging caused by various and varied environmental factors and corresponds more particularly to photoaging due to exposure to the sun but also to pollutants, tobacco, etc. It results in changes such as relatively thick wrinkles, the appearance of spots and the formation of “parchment” skin.

Intrinsic or chronobiological aging corresponds to normal or physiological aging linked to age and is expressed in particular by a slowing of the renewal of epidermal cells and the appearance of fine lines or wrinkles. With age, therefore, the skin loses a portion of the water retained in the extracellular matrix, and then undergoes an aging process that results in an increase in fibrosis and a decrease in the fiber elasticity. During the aging process, a modification of skin structure and functions are observed.

The principal clinical signs of skin aging are the appearance of fine lines and deep wrinkles, which obviously increase with age. Furrows and wrinkles are marked; the skin becomes hollow and loses its firmness; on the surface, the skin loses its radiance.

Among extrinsic factors, as indicated, mention may be made of exposure to temperature and humidity variations or exposure to pollutants, UV rays, etc. Among intrinsic factors affecting skin tone radiance, mention may be made of stress, fatigue, hormonal changes, dehydration of the epidermis or change in skin barrier function, i.e., all factors associated with chronobiological aging.

These extrinsic and intrinsic factors tend to blur the complexion, to make it inhomogeneous, dull, waxy, to promote or even aggravate the presence of skin imperfections, wrinkles, fine lines, pigment spots, age spots. These changes in the complexion, often multifactorial in origin, are an increasingly common cause of consultations in beauty care centers or with dermatologists.

In recent years, various hydrolysates obtained by enzymatic hydrolysis of plant and animal proteins with various proteolytic enzymes have been studied for theft physiological activities, and they have been used for various processed foods, seasonings, shampoos, cosmetics, etc,

For example, KR101841088 describes an oyster extract obtained by proteolysis of oyster meat via added proteases, at acidic pH. Said extract enriched with peptides derived from enzymatic proteolysis by exogenous proteases is used to treat wrinkles.

The Applicant demonstrated that, surprisingly, an extract of oyster meat, obtained by alkaline hydrolysis, therefore without enzymatic proteolysis or the addition of exogenous proteases, exhibited highly advantageous properties for the treatment of the signs of skin aging and for stimulating skin cell growth.

The compositions according to the invention can be used in topical preparations, in the field of dermatology or cosmetology, for the purpose of preventing or treating the signs of aging for which it will be necessary to reduce wrinkles, to combat photoinduced or non-photoinduced skin aging, to revive epidermal and dermal cell activity, and thus to firm the skin, to increase its elasticity, to prevent or treat spots, or to stimulate wound healing.

Therefore, the present invention relates, according to an embodiment, to an oyster extract obtained by alkaline hydrolysis of oyster meat.

The term “oyster”, as used herein, refers to a bivalve mollusk of the family Ostreidae.

More particularly, reference is made to the species Pynodonta, Crassostrea or Ostrea.

The genus Pycnodonta includes the species of deep-sea oysters, they live in places that are continually immersed (up to 2000 m). They have a very round shell made of vacuoles.

The Crassostrea are oysters of the intertidal zone (part of the coastline discovered at each tide). Reproduction takes place outside the shell, at random encounters between eggs and spermatozoa.

The genus Ostrea lives in areas that are always immersed or only occasionally exposed and has a different mode of reproduction: fertilization takes place inside the shell, then the larvae are discharged to the outside world.

An oyster consists of two valves, or shells, enclosing a fleshy part. This fleshy part comprises, the mantle, the gills, the milt depending on the season and the adductor muscle.

In the context of the present invention, reference is made to an extract of “oyster meat”, which is an extract obtained from the fleshy part, that is to say, the whole comprising the mantle and the gills, the milt if applicable, and all or part of the adductor muscle.

Preferentially, the oyster meat used comprises the milt of the oyster.

The extract according to the invention is an extract obtained by alkaline hydrolysis of oyster meat.

The oyster meat can be fresh or frozen, whole, cut or ground and then subjected to an alkaline hydrolysis step.

The term “alkaline hydrolysis” of oyster meat, as used herein, means that the oyster meat is incubated with an alkaline compound, at a pH comprised between 8 and 13, particularly between 9 and 12. In particular, this hydrolysis is carried out without the addition of any external enzyme, particularly without the addition of external proteolytic enzyme.

The alkaline hydrolysis according to the invention is not an enzymatic proteolysis given the absence of the addition of protease and the pH at which this hydrolysis is carried out.

The alkaline compound can be any base, in particular a strong base, such as NaOH or KOH, or (Ca(OH)₂) or NH₄OH, in an amount and at a concentration allowing the desired pH to be obtained.

According to a preferred embodiment, the extract according to the invention is obtainable by a process according to the invention described hereinbelow. Such a process for the preparation of an extract according to the invention comprises a step of maceration of oyster meat, in particular of meat with milt, in alkaline medium at a pH comprised between 8 and 13, particularly between 9 and 12, more particularly about 11.

Particularly, the oyster meat, raw fresh or frozen, optionally reduced to pieces and/or ground, is mixed with a base, preferably a base, in particular a strong base and the whole is left to incubate, preferably at room temperature, for 2 to 12 hours, in particular for 3 to 9 hours, more particularly about 6 hours.

The oyster meat is first drained in order to eliminate excess vegetation water, and/or mucus, and to avoid the dilution of the alkaline hydrolysis medium. Drained oyster meat means here oyster meat containing between 80 and 90% by weight water, i.e., oyster meat that is native but free of vegetation water or mucus.

The incubation mixture can be stirred by any suitable means, either continuously or intermittently.

The chosen base can be NaOH, KOH, (Ca(OH)₂) or NH₄OH, in an amount and at a concentration sufficient to reach the desired pH.

Typically, the base, such as NaOH, NH₄OH, (Ca(OH)₂) or KOH, for example, will have a concentration of 0.5 to 2 M, particularly 1 M.

According to another particular embodiment of the invention, alkaline hydrolysis is carried out under stirring or statically, at a temperature comprised between 2° C. and 37° C., in particular between 2° C. and 25° C., more particularly between 2° C. and 20° C., even more particularly between 2° C. and 15° C., or even between 2° C. and 10 C.

It can be assisted by ultrasound or by extrusion.

The duration of hydrolysis can be comprised between 1 minute and 48 hours, more particularly about 3 to 10 hours, and even more particularly about 6 hours. The extraction can be repeated once, or even 2 to 3 times.

The ratio of base volume to oyster meat mass can vary from 15:1 to 2:1, particularly from 15:1 to 5:1, more particularly of the order of 10:1.

This ratio will be variable as a function of the concentration of the base and the amount of residual water contained in the oyster meat. The ratio is not critical in itself and will be determined as a function of these parameters in order to obtain a pH of the mixture to be incubated comprised in the range between 8 and 13, particularly between 9 and 12, more particularly about 11.

The invention is thus aimed at an oyster extract obtained by alkaline hydrolysis of oyster meat at a pH comprised between 8 and 13, particularly between 9 and 12 and without the addition of any external enzyme, particularly without the addition of external proteolytic enzyme.

The extract according to the invention is an extract of oyster meat freed of its mucus and/or vegetation water.

After the step of incubation in an alkaline medium as indicated above, the mixture is neutralized by adding an acid in order to obtain a pH of the medium of the order of 4 to 7, particularly from 4 to 6, more particularly from 4 to 5 The neutralization can be carried out by any suitable acid, weak or strong, for example selected from citric acid, sulfuric acid, acetic acid or any other suitable acid.

The mixture is neutralized or then subjected to a liquid/solid separation step in order to recover the clear liquid phase free of suspended particles.

Any suitable technique such as filtration, centrifugation to remove pieces, particles and cellular debris is suitable. Typically, the liquid/solid separation step is carried out to remove particles larger than 20 μm.

According to another particular embodiment of the invention, the solid phase is then separated by centrifugation or filtration in order to recover a clear liquid phase free of particles. Filtration can be carried out on filter paper or filter plate with a cut-off comprised between 5 and 20 μm, particularly between 10 and 20 μm. Suitable filters or filter plates can be selected from the K series depth filtration plate range from Pall®. Such plates are composed of a balanced mixture of cellulose fibers, diatomaceous earth, and perlite, which creates a well-defined matrix. Pall® K-series plates of type K300 to K900 can thus be selected. The liquid phase representing the extract can be more or less concentrated, up to a dry extract.

The clear liquid phase obtained constitutes an extract according to the invention.

The dry matter of an extract according to the invention thus obtained can be comprised between 2 and 15%, particularly between 2 and 12%, particularly around 10%, in % by mass. This dry matter of the extract comprises the organic matter of the hydrolyzed oyster but also the added compounds, base for hydrolysis and acid for neutralization.

In another embodiment of the invention, a carrier can also be added during the concentration step in order to obtain an extract containing 1 to 75% dry extract or dry matter.

The carrier may be maltodextrin, lactose, silica, glycerin, a glycol, or any other carrier that is cosmetically acceptable and solubilizes the extract, preferentially of bio-based origin such as, for example, bio-based glycols (1,2-pentanediol; 1,3-butanediol; 1,3-propanediol, etc.), and also hydrotropes such as alkyl glycosides (Sepiclear, Apyclean, APXC4, etc.).

According to a particular embodiment of the invention, the extract can be decolorized, for example on activated carbon.

The extract thus obtained can be used as is or more or less concentrated, or even dried in powder form with or without drying carrier.

The extract, without carrier, can thus be concentrated under vacuum up to a mass rate of dry matter comprised between 5 and 30%, preferentially between 5 and 20% and more particularly still between 5 and 15%, or even more particularly around 10%.

Concentration can be carried out thermally (at a temperature preferentially below 50° C. and under vacuum) in order to increase Brix degrees and stabilize with respect to microbiological contaminations.

The extract can also be dried alone or on a carrier (for example maltodextrin, starch or lactose).

Advantageously, the concentrated extract corresponds to an extract corresponding to 1.5 to 8 grams of extract per 1 gram of oyster meat, more particularly between 2 and 6 grams of extract per 1 gram of oyster meat, more particularly still about 3 to 5 grams of extract per 1 gram of oyster meat, more particularly about 4 grams of extract per 1 gram of oyster meat. Here, it is a matter of drained oyster meat.

The process for manufacturing the extract is characterized in that no exogenous enzyme is added to the mixture. In particular, no protease is added to the mixture of oyster meat and base.

Hydrolysis of the organic matter of the oyster thus consists of alkaline hydrolysis and the organic matter is thus reduced to its essentially soluble organic and mineral components. In this alkaline hydrolysis, the combination of the movement of water, its temperature, and its alkalinity accelerates the process of dissolution and decomposition of the tissues, which after a few hours of treatment, disappears giving a colored liquid rich in amino acids, peptides, carbohydrates, fatty acid soaps, nucleotides, salts, etc.

According to a particular embodiment, the oyster extract according to the invention comprises in % by weight, in relation to the dry matter of the extract, from 1% to 10% free amino acids; from 3% to 12% proteins or peptides, from 0.005% to 0.05% total sugars and from 0.1% to 1% polyphenols.

The amount of free amino acids, in % by weight, in relation to the dry matter of the extract, may be comprised between 1 and 8%; that of proteins or peptides, in % by weight, in relation to the dry matter of the extract, between 5 and 10%; that of total sugars, in % by weight, in relation to the dry matter of the extract, between 0.01 and 0.05% and that of polyphenols, in % by weight, in relation to the dry matter of the extract, between 0.1 and 0.5%. The % are expressed in % by weight in relation to the dry matter of the extract. As indicated above, the extract matter comprises the organic matter of the oyster but also more or less compounds added for hydrolysis (i.e., base) and for neutralization (i.e., acid).

It is also an object of the present invention to provide a dermatological or cosmetic composition comprising an oyster extract according to the present invention and a dermatologically or cosmetically acceptable excipient.

The pharmaceutical or cosmetic composition according to the invention comprises an extract according to the invention as defined above mixed with at least one pharmaceutically or cosmetically acceptable excipient. It is preferably a topical composition for the skin, in particular for the skin of the face and/or the skin of the hands. In the present invention, “pharmaceutically or cosmetically acceptable” means that which is useful in the preparation of a pharmaceutical or cosmetic composition, which is generally safe, non-toxic and neither biologically nor otherwise undesirable and which is acceptable for pharmaceutical or cosmetic use, and in particular dermatological or cosmetic use, in particular by topical application. The compositions according to the invention are advantageously intended for topical application, in particular to the skin. Preferably, the cosmetically acceptable excipients are suitable for topical administration. In particular, the acceptable excipients ensure good stability, pleasant texture and feel. They may also be for example formulating agents or additives of known and conventional use in cosmetics: mention may be made of surfactants, dyes, preservatives, fragrances, film-forming agents, thickeners, etc.

The compositions according to the invention may thus be in the forms that are usually known for topical administration, i.e., in particular lotions, foams, gels, dispersions, emulsions, sprays, serums, masks or creams, with excipients allowing in particular skin penetration in order to improve the properties and accessibility of the active principle.

Advantageously, it will be a cream. These compositions generally contain, in addition to the extract according to the present invention, a physiologically acceptable medium, generally based on water or solvent, for example alcohols, ethers or glycols. They may also contain surfactants, complexing agents, preservatives, stabilizers, emulsifiers, thickeners, gelling agents, humectants, emollients, trace elements, essential oils, perfumes, dyes, mattifying agents, chemical or mineral filters, moisturizing agents.

According to an embodiment, the dermatological or cosmetic composition according to the invention is characterized in that the amount of oyster extract according to the invention is comprised between 0.01% and 10% by weight in relation to the total weight of the composition, more particularly between 0.05% and 5% by weight in relation to the total weight of the composition. This percentage may vary according to the dry matter content of the extract and its concentration.

Typically, the dermatological or cosmetic composition according to the invention is characterized in that it is in an oral or topical form, preferentially in topical form.

The object of the present invention is aimed at the cosmetic, particularly non-therapeutic, use of an extract according to the invention or of a dermatological or cosmetic composition containing said extract according to the present invention or of this dermatological or cosmetic composition according to the invention to combat the signs of skin aging.

The cosmetic use of the extract according to the present invention or the cosmetic or dermatological composition according to the invention is more particularly intended to restore material to the skin, reinforce its firmness and visibly reduce marked wrinkles and deep furrows.

The present invention also relates to a method for combating the signs of skin aging comprising the administration, preferably topical, of an effective amount of an extract according to the invention or of a cosmetic or dermatological composition according to the invention to a person in need thereof.

Another object of the present invention relates to a dermatological composition intended to accelerate skin repair in order to restore the integrity and quality of the skin comprising as dermatological or cosmetic active principle the extract according to the invention and further comprising at least one dermatologically or cosmetically acceptable excipient.

According to an embodiment, the present invention is thus aimed at the use of a cosmetic composition for the prevention and/or treatment of skin disorders, said cosmetic composition comprising, as active principle, an oyster extract according to the present invention.

In an embodiment, the use according to the invention is characterized in that skin disorders are manifested by changes in texture, color, transparency of the skin, irritation and the appearance of wrinkles.

More particularly, the skin disorders are the result of environmental stress.

According to a particular embodiment, the environmental stress is caused by the sun, tobacco.

According to an alternative, the use according to the invention is characterized in that the composition is in oral or topical form, preferentially in topical form.

In a preferential manner, the topical form is selected from the group comprising creams, gels, ointments and sprays.

In an embodiment where the use is aimed at an oral form, the latter is characterized in that this oral form is selected from the group comprising tablets, capsules and powders for oral suspensions. The composition according to the present invention may be administered orally or by any other pharmaceutical route of administration. The compositions according to the present invention may be formulated for administration to mammals, including humans. These compositions are prepared in such a way that they can be administered orally, sublingually, subcutaneously, intramuscularly, intravenously or transdermally. In this case, the active ingredient(s) may be administered in unit dosage forms, mixed with conventional pharmaceutical carriers, to animals or to humans.

In the case of a food supplement or a drug, the following dosage forms may be envisaged: capsules, oral tablets, chewable tablets, effervescent tablets, lozenges, pills, powders, granules, oral solutions or suspensions, and sublingual and buccal forms of administration. The preferred dosage form is the capsule. When a solid composition in tablet form is prepared, the extract according to the invention is mixed with a pharmaceutical vehicle such as gelatin, starch, lactose, magnesium stearate, talc, gum arabic, silica or the like. A capsule preparation is obtained by mixing the extract according to the invention with a diluent (optional step) and pouring the mixture obtained into soft or hard capsules. A preparation in syrup or elixir form may contain the extract according to the invention together with a sweetener, a flavoring agent and a suitable dye. Water-dispersible powders or granules may contain the extract according to the invention mixed with suspending agents, as well as with taste regulators or sweeteners.

Advantageously, the composition according to the present invention is intended for oral administration.

Another object of the present invention is a composition according to the invention for use as a medicinal product.

Another object of the present invention is aimed at an extract or composition according to the present invention for use in stimulating cell growth and promoting wound healing.

Thus, the invention is also aimed at a dermatological composition comprising an oyster extract according to the invention and a dermatologically acceptable excipient, for use in the treatment of wound healing and skin irritation.

Indeed, it has been demonstrated that an extract according to the invention significantly promotes the synthesis of procollagen by fibroblasts, which is one of the fibrillar proteins constituting an important part of the connective tissue involved in the healing of superficial skin wounds and in particular in scars following superficial skin injury, inflammation or irritation.

Indeed, skin repair begins in the 24 hours following skin injury, inflammation or irritation, by the migration and induction of fibroblasts, and the proliferation of endothelial cells. In 3 to 5 days, the specialized granulation tissue characteristic of healing appears. Its histological appearance is characterized by a proliferation of fibroblasts and new thin-walled capillaries, often mixed with inflammatory cells, chiefly macrophages. In the granulation tissue gradually accumulates more fibroblasts, which deposit more collagen, resulting in the formation of a scar. The scar is remodeled over time.

The invention is further aimed at a method of cosmetic, in particular non-therapeutic, treatment of the skin intended to improve, prevent or treat skin disorders comprising, or consisting of, the application to the skin of a cosmetic composition according to the invention.

The method of cosmetic treatment according to the invention is characterized in that the improvement of skin disorders comprises the improvement of the signs associated with skin aging, in particular an effect selected from: the improvement of wrinkles, the improvement of skin firmness and the obtaining of a skin tensor effect.

The method of cosmetic skin treatment according to the invention is also characterized in that the improvement of skin disorders associated with skin aging comprises the treatment of age spots and the lightening of the complexion.

### EXAMPLE 1

### Oyster Meat Collection

Collection environment: in a closed room.

Material to be collected=milky oysters with “clean” shells (brush and rinse the shells well before opening);

Equipment and accessories=freezer bags +ice tray that will be used to put the bags of oyster meat.

The oysters are opened and the meat (mantle, muscle, milt) is removed and placed in a bag on ice.

Once closed, the bag is weighed and stored for up to another 30 minutes on the ice before placing the bag in the freezer at −18° C. or −20° C.

### EXAMPLE 2

### Obtaining the Extract by Alkaline Hydrolysis and Characterization

The extract resulting from alkaline hydrolysis is made from frozen “milky” oysters, kept in dry ice as soon as they are extracted from the shell.

The hydrolytic approach chosen is the alkaline route, and more particularly a 1 M NaOH solution.

The oyster meat (approx. 200 g drained and thus freed of vegetation water) is mixed with 200 ml of a 1 M NaOH solution in order to obtain a pH value of the solution of about 10 to 11.

After a contact time of 6 hours under stirring at room temperature and in darkness, the mixture is then neutralized with citric acid to obtain a pH of about 4.7.

The mixture is filtered on a cellulose filter and then concentrated under vacuum so as to obtain 4 g of extract per 1 g of fresh oyster. The extract thus obtained has a dry matter content of 10%.

The extract obtained has the following characteristics:

| | Amount (in % by weight in |
|---|---|
| | relation to the dry matter |
| Oyster extract | of the extract) |
| Protein | 7.8% |
| Total polyphenols | 0.3% |
| Total sugars | 0.01% |
| Free amino acids | 2% |

### Protein Determination

Total proteins are tested according to the Lowry method. For this colorimetric assay, a standard curve OD750 nm=f(amount of proteins) is prepared. The reference protein used in this experiment is bovine serum albumin (BSA). The determination of the total amount of proteins in each extract is obtained using the same protocol as that carried out for the standard range.

### Polyphenol Determination

Total polyphenols are evaluated using the Folin-Ciocâlteu reagent method. For this colorimetric assay, a standard curve OD720 nm=f is prepared using increasing concentrations of a reference molecule. The reference polyphenol used in this experiment is gallic acid. The determination of the total amount of polyphenols in each extract is obtained using the same protocol as that carried out for the standard range.

### Sugar Determination

Total sugars are evaluated using the sulfuric acid-anthrone hydrolysis method. For this colorimetric assay, a standard curve OD578 nm=f is prepared from increasing concentrations of a reference molecule. The reference sugar used in this experiment is glucose. The determination of the total amount of sugars in each extract is obtained using the same protocol as that performed for the standard range.

### EXAMPLE 3

### Studies of Biological Properties of the Extract

The extract used is that obtained according to example 1, with a dry matter content of 10%.

It is used after dilution in ultrapure water. The concentrations of the extract are: 0.5%, 1.5% and 4.5% w/v (in grams of extract per 100 mL of water).

Bench test with collagen microbeads/Measurement of the tensor effect. In this model, freeze-dried 8-mm-diameter collagen discs are used. The contraction of these discs in response to different treatments is measured by image analysis. The more the surface area of the collagen discs decreases, the greater the tensor effect of the active agents.

The reference product used in this study is bovine serum albumin at 100 mg/ml.

The 8-mm-diameter collagen discs are soaked with 40 μl ultrapure water (control), the reference product and increasing concentrations of the test product.
RESULT Tensor effect
   For extract at 1.5%/result+49.4%;
   For Extract at 4.5%/result+56.5%

Tests on normal human skin cell cultures/melanin inhibition:

This study model uses normal human melanocytes obtained from the foreskin of a 4-year-old donor. The melanocytes are isolated and cultured in a monolayer until confluence. The cells are then incubated for 72 H in the absence (control) or presence of a reference product (positive control) or increasing concentrations of the product to be tested.

The reference product (positive controls) is kojic acid at 250 μM.

At the end of the incubation period, the amount of intracellular melanin is evaluated in the cell lysate by spectrophotometric measurement at 405 nm.

RESULT Melanin Inhibitory Effect:

Extract at 0.5%; Result: −17.1%.

### Stimulatory Effect on the Production of Procollagen Type I

This study model uses normal human fibroblasts obtained from a 68-year-old female donor. The fibroblasts are isolated and cultured in a monolayer until confluence. The cells are then incubated for 48 H in the absence (control) or presence of a reference product (positive control) or increasing concentrations of the product to be tested.

The reference product (positive controls) is TGF-Beta at 1 ng/ml and 10 ng/ml.

After 48 H of incubation, the amount of procollagen type I produced in the culture medium is evaluated using a sensitive and specific ELISA kit.

### RESULT: Stimulation of Procollagen Type I Production:

Extract at 0.015%; Result+18%.

These results confirm the positive effect of an extract according to the invention in the treatment of wound healing following superficial skin injury or irritation.
Enzyme Tests

### Type I Collagenase Inhibitory Effect

This assay system is carried out with purified type I collagenases produced by Clostridium histolyticum, and a specific substrate detectable by a colorimetric method. The consumption of this substrate by type I collagenases is monitored by measurement of the optical density (OD) with a spectrometer at a wavelength of 405 nm every 3 minutes. The incubation period for these measurements is 45 minutes.

A reference control (namely EDTA at 2.5 mM) was used to verify the modulation capacity and in particular the inhibition capacity of the test enzymes.

### RESULT on Type I Collagenase Inhibition

Extract at 0.15%; result: −67.3%.

Extract at 0.5%; result −82.2%.

Extract at 1.5%; result −91.1%.

### Inhibitory Effect on Total Collagenases

This assay system is carried out with semi-purified collagenases derived from the culture medium of a normal human fibroblast culture, and a specific substrate detectable by a colorimetric method. The consumption of this substrate by the total collagenases is monitored by measurement of the optical density (OD) with a spectrometer at a wavelength of 405 nm every 3 minutes. The incubation period for these measurements is 45 minutes.

A reference control (namely EDTA at 2.5 mM) was used to verify the modulation capacity and in particular the inhibition capacity of the test enzymes.

### RESULT on Total Collagenase Inhibition

Extract at 0.5%; result −18.7%.

Extract at 1.5%; result −44.7%.

### CONCLUSION

The extract (at 10% dry matter), and this from the dose of 1%, shows a tensor effect which on the surface of the skin can bring a tension effect so as to erase wrinkles. The stimulation of the production of type I procollagen on fibroblast culture shows that the extract derived from alkaline hydrolysis of oyster meat improves the content of the extracellular matrix of the skin tissue by increasing the fibers associated with skin firmness. This capacity also makes it possible to envisage wrinkle filling. On the strength of this hypothesis, we rule out any decoy effect (the degradation of collagen fibers stimulates collagen production by reaction, therefore any stimulation of collagenase activity could claim to indirectly stimulate collagen production).

This performance is reinforced by the capacity of the extract to stop the degradation of collagen by inhibition of the type I collagenase enzyme and more generally of all collagenases, any decoy effect is thus ruled out, we observe a real biological capacity of the extract to stimulate type I procollagen. Finally, the extract limits melanin production of from the 0.5% dose, this property helps to limit age spots, but also to lighten the complexion.

1. An oyster extract obtained by alkaline hydrolysis of oyster meat at pH comprised between 8 and 13, particularly between 9 and 12 and without the addition of any external enzyme, particularly without the addition of any external proteolytic enzyme.

2. The oyster extract as claimed in claim 1 comprising in % by weight, in relation to the dry matter of the extract, from 1% to 10% free amino acids; from 3% to 12% proteins or peptides, from 0.005% to 0.05% total sugars and from 0.1% to 1% polyphenols.

3. A dermatological or cosmetic composition comprising an oyster extract as claimed in claim 1 or 2 and a dermatologically or cosmetically acceptable excipient.

4. The composition as claimed in claim 3, characterized in that the amount of oyster extract is comprised between 0.01% and 10% by weight in relation to the total weight of the composition.

5. The composition as claimed in one of claim 3 or 4, characterized in that the composition is in oral or topical form, preferentially in topical form.

6. The cosmetic composition as claimed in one of claims 3 to 5 for use in the prevention and/or treatment of skin disorders, said cosmetic composition comprising, as active principle, an oyster extract as claimed in one of claim 1 or 2 in combination with a cosmetically acceptable excipient.

7. The composition for its use as claimed in claim 6, characterized in that skin disorders are manifested by changes in the texture, color and transparency of the skin, and the appearance of wrinkles.

8. The composition for use as claimed in one of claim 6 or 7, characterized in that the skin disorders are the result of environmental stress.

9. The composition for use as claimed in claim 8 characterized in that environmental stress is caused by the sun, tobacco.

10. The composition for use as claimed in one of claims 6 to 9, characterized in that the composition is in oral or topical form, preferentially in topical form.

11. The composition for use as claimed in claim 10, characterized in that the topical form is selected from the group consisting of creams, gels, ointments and sprays.

12. The composition for use as claimed in claim 10, characterized in that the oral form is selected from the group comprising tablets, capsules and powders for oral suspensions.

13. The composition as claimed in one of claims 3 to 5 for use in improving the signs associated with skin aging, in particular selected from: the improvement of wrinkles, the improvement of skin firmness and the obtaining of a skin tensor effect, the treatment of age spots and lightening of the complexion.

14. A dermatological composition comprising an oyster extract as claimed in one of claim 1 or 2 and a dermatologically acceptable excipient, for use in the treatment of wound healing and skin irritation.
